(19) United States
(12) Patent Application Publication
(10) Pub. No.: US 2015/0105815 A1
(43) Pub. Date: Apr. 16, 2015

(54) HIGH PRESSURE TEAR RESISTANT BALLOON

(71) Applicant: Boston Scientific Scimed, Inc., Maple Grove, MN (US)

(72) Inventors: Daniel J. Horn, Shoreview, MN (US); Aaron Khieu, Maple Grove, MN (US); Jeffry Johnson, Crystal, MN (US); Paul O'Flynn, Galway (IE)

(73) Assignee: Boston Scientific Scimed, Inc., Maple Grove, MN (US)

(21) Appl. No.: 14/062,367

(22) Filed: Oct. 24, 2013

Related U.S. Application Data
(60) Provisional application No. 61/891,204, filed on Oct. 15, 2013.

(51) Int. Cl. A61M 29/02

(52) U.S. Cl. CPC A61M 29/02
USPC 606/192

(57) ABSTRACT

An expandable medical balloon comprising an inner layer formed of a polymer material having a first flexural modulus, an intermediate layer formed of a material having a second flexural and an outer layer formed of a material having a third flexural modulus, wherein the flexural modulus of the inner layer and outer layer is 50,000 psi to about 80,000 psi, the flexural modulus of the intermediate layer is about 130,000 to about 230,000 psi and the calculated burst strength of the balloon determined on a 90 degree bend in the balloon is about 35,000 psi or higher.

### CROSS-REFERENCE TO RELATED APPLICATIONS

This application claims priority to U.S. Patent Provisional Application No. 61/891,204, filed Oct. 15, 2013, the entire contents of which are hereby incorporated herein by reference.

### BACKGROUND OF THE INVENTION

The present invention relates to the field of expandable medical balloons, particularly those employed for dilatation and delivery of medical devices.

Expandable medical balloons are employed in a variety of medical procedures including plain old balloon angioplasty (POBA) as well as for delivery of medical devices to the treatment site such as stent delivery.

Medical applications wherein a balloon is employed intraluminally such as for POBA and stent delivery can be demanding applications due to the extremely small vessels, and the tortuous and long distances the catheter may travel to the treatment site.

For applications where the lesion in the vessel is highly resistant and focalized, such as at the center of a bend in the body vessel, a reduction in burst pressure can be seen with single layer and with dual layer balloons, as well as micro tearing on the exterior of the balloon as a result of localized stress.

Such issues can be even more pronounced when dilatation and/or stenting is being done in the peripheral vasculature.

Compounding the issue even more is that it is typically desirable that the balloon be thin walled, while still maintaining high strength as most commonly measured by hoop strength or pressure at burst, be relatively inelastic, and have predictable inflation properties.

Inelasticity is desirable to allow for easy control of the diameter, but some elasticity is desirable to enable the surgeon to vary the balloon's diameter as required to treat individual lesions. Suitably, small variations in pressure should not cause wide variation in balloon diameter.

It can be difficult to achieve an excellent balance of properties with a single polymer material. Therefore, a variety of polymer blends and multiple layer polymer balloons have been developed over the years.

There remains a need in the art, however, for an expandable medical balloon having an excellent balance of physical properties.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### SUMMARY OF THE INVENTION

In one aspect, the present invention relates to an expandable medical balloon comprising an inner layer formed of a poly(ether-block-amide) copolymer, an intermediate layer formed of a polyamide and an outer layer comprising a polymeric material having a flexural modulus that is less than the intermediate layer, wherein the calculated burst strength of the balloon determined on a 90 degree bend in the balloon is about 35,000 psi or higher.

In another aspect, the present invention relates to an expandable medical balloon comprising, an inner layer formed of a poly(ether-block-amide) copolymer, an intermediate layer formed of a polyamide and an outer layer comprising a polymeric material having a flexural modulus that is less than the flexural modulus of the intermediate layer outer layer, wherein the compliance is about 0.25% radial growth/atmosphere to about 0.50% radial growth/atmosphere from nominal pressure to rated burst pressure.

In another aspect, the present invention relates to an expandable medical balloon comprising an inner layer formed of a polymer material having a first flexural modulus, an intermediate layer formed of a material having a second flexural and an outer layer formed of a material having a third flexural modulus, wherein the flexural modulus of the inner layer and outer layer is 50,000 psi to about 110,000 psi, the flexural modulus of the intermediate layer is about 130,000 to about 230,000 psi and the calculated burst strength of the balloon determined on a 90 degree bend in the balloon is about 35,000 psi or higher.

In another aspect, the present invention realtes to an expandable medical balloon comprising an inner layer formed of a poly(ether-block-amide) copolymer, an intermediate layer formed of a polyamide and an outer layer comprising a polymeric material having a durometer that is less than the outer layer, wherein the calculated burst strength of the balloon determined on a 90 degree bend in the balloon is about 35,000 psi or higher.

These and other aspects, embodiments and advantages of the present disclosure will become immediately apparent to those of ordinary skill in the art upon review of the Detailed Description and Claims to follow.

### BRIEF DESCRIPTION OF THE DRAWINGS

FIG. 1 is a table including a comparison of Rockwell hardness scale, Shore A hardness scale and Shore D hardness scale.

FIG. 2 is a longitudinal cross-section of a balloon having a tri-layer configuration according to the invention.

FIG. 3 is a radial cross-section taken at section 3-3 in FIG. 2.

FIG. 4 is a longitudinal cross-section of a catheter assembly equipped with a balloon according to the invention.

FIG. 5 is a side view of an expandable medical balloon with a stent disposed thereon.

FIG. 6 is a side view illustrating a balloon burst test wherein the balloon calculated burst strength is determined on a 90 degree balloon bend.

FIG. 7 is a is a graph illustrating the burst strength of a dual layer balloon versus the burst strength of a tri-layer balloon according to the invention wherein the burst strength is determined on a 90 degree bend in the balloon.

FIG. 8A is a graph illustrating the compliance of a 5×20 mm dual layer balloon versus the compliance of a 5×20 mm tri-layer balloon according to the invention as determined by its radial growth in mm from nominal pressure to rated burst pressure.

FIG. 8B is a graph illustrating the compliance of a 5×120 mm dual layer balloon versus the compliance of a 5×120 mm tri-layer balloon according to the invention as determined by its radial growth in mm from nominal pressure to rated burst pressure.

FIG. 9A is a graph illustrating the compliance of a 6×20 mm dual layer balloon versus the compliance of a 6×20 mm tri-layer balloon according to the invention as determined by its radial growth in mm from nominal pressure to rated burst pressure.

FIG. 9B is a graph illustrating the compliance of a 6×100 mm dual layer balloon versus the compliance of 6×100 mm tri-layer balloon according to the invention as determined by its radial growth in mm from nominal pressure to rated burst pressure.

FIG. 10A is a graph illustrating the compliance of a 12×20 mm dual layer balloon versus the compliance of a 12×20 mm tri-layer balloon according to the invention as determined by its radial growth in mm from nominal pressure to rated burst pressure.

FIG. 10B is a graph illustrating the compliance of a 12×80 mm dual layer balloon versus the compliance of 12×80 mm tri-layer balloon according to the invention as determined by its radial growth in mm from nominal pressure to rated burst pressure.

### DETAILED DESCRIPTION OF THE INVENTION

While embodiments of the present disclosure may take many forms, there are described in detail herein specific embodiments of the present disclosure. This description is an exemplification of the principles of the present disclosure and is not intended to limit the disclosure to the particular embodiments illustrated.

The present invention relates to an expandable medical balloon having at least three layers including an inner softer, more elastic layer, an intermediate harder, less elastic layer and another outer softer, more elastic layer. Suitably, the softer, more elastic inner and outer layers is formed from a material which also has a lower tensile set (see ASTM D412). This lower tensile set material forming the inner layer provides for improved refoldability making withdrawal easier after a procedure is complete.

Suitably, the inner and outer layers are formed from a polymer material having a flexural modulus of about 40,000 psi to about 110,000 psi, suitably about 50,000 psi to about 80,000 psi and more suitably about 55,000 psi to about 75,000 psi.

Suitably, the inner and outer layers are formed from a polymer material having a durometer as measure on the Shore D hardness scale of about less than about 75D, more suitably less than about 70D, with a range of about 25D to about 75D, more suitably about 25D to about 70D. In some embodiments, the range is about 50D to about 75D, more suitably 50D to about 70D.

The more inelastic, harder intermediate layer is formed from a polymer material having a flexural modulus of about 130,000 to about 230,000 psi, more suitably about 130,000 psi to about 210,000 psi.

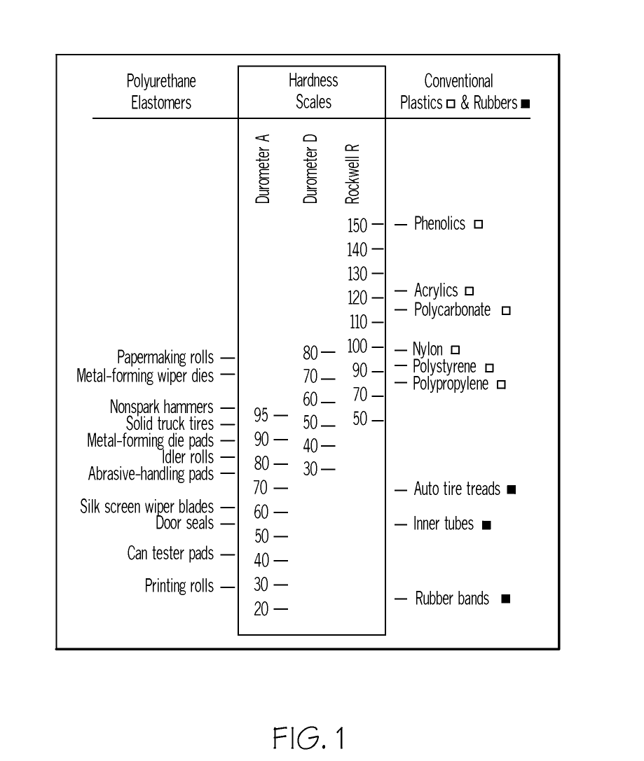

The outer layer may have a durometer as measured on the Rockwell hardness scale of between about 60 and about 115, more suitably about 70 to about 115, and most suitably about 80 to about 115, although this range may vary. The durometer of the outer layer based on the Shore D hardness (ASTM D2240) scale is suitably greater than about 75D, and more suitably greater than about 80D. A comparison of Shore A, Shore D and Rockwell hardness is shown in FIG. 1. FIG. 1 is reproduced from http:www/calce.umd.edu/general/Facilities/Hardness_ad.htm. As can be seen from the scale, nylon has a Shore D harness of 80 or greater and a Rockwell hardness of greater than about 95. These numbers are approximated from the scale.

Vestamid® L polyamide 12 series, found to be useful herein, have a Shore D hardness of about 68-, and typically about 74.

Shore D hardness values of PEBAX® 6333, 7033 and 7233 can be found at http://www.pebax.com/sites/pebax/en/properties/mechanical_properties1.page and are reproduced below in table 1. The standard deviation for these measurements is typically about +/−3. The standard used for these measurements is ASTM standard D 790 or ISO 178.

TABLE 1

| | Shore A | | Shore D | |
|---|---|---|---|---|
| | Hardness | | Hardness | |
| Pebax ® Grade | Instantaneous | After 15 s | Instantaneous | After 15 s |
| 4033 | 90 | 89 | 41 | 34 |
| 5533 | — | — | 54 | 50 |
| 6333 | — | — | 64 | 58 |
| 7033 | — | — | 69 | 61 |
| 7233 | — | — | 69 | 61 |

In one embodiment, the inner layer is formed from a poly(ether-block-amide), the intermediate layer is formed from a polyamide or nylon, and the outer layer is formed from a poly(ether-block-amide). In a preferred embodiment, the outer layer is nylon 12, formed from laurolactam. Nylon 12 is available from Degussa-Hüls AG, North America under the tradename of Vestamid® L2101F. Degussa's national headquarters are located in Düsseldorf, Germany. Nylon 12 is available from a variety of polymer manufacturers. Grilamid® L25 is another nylon 12 commercially available from EMS-Grivory.

Poly(ether-block-amide copolymers are available from Arkema, North America under the tradename of Pebax®. Arkema's headquarters are located in Philadelphia, Pa. Specific grades of Pebax® useful herein include, but are not limited to, 6333 and 7033, and 7233. In a specific embodiment, the inner layer is formed from Pebax® 7033 and the outer layer is formed from Pebax® 7233.

Other materials such as polyurethane elastomers, for example Tecothane® polyurethanes available from Noveon, Inc. in Cleveland, Ohio, find utility for use as the inner, softer layer. A specific example is Tecothane® TT-1074A.

The balloons range in diameter size from about 3 mm to about 12 mm.

Suitably, the inner layer and the intermediate layer comprise between about 30% to about 50% of the wall thickness of the balloon and the outer layer comprises no more than about 20% of the wall thickness of the balloon and more suitably no more than about 15% of the wall thickness of the balloon.

The balloons according to the invention have a 2× wall thickness from about 0.0015″ to about 0.0040″, suitably about 0.0017″ to about 0.0037″ and a diameter from about 3 to about 12 mm.

The inner layer provides at least 10%, and in some embodiments at least 20% of the burst strength of the balloon. Optionally, a lubricious coating may be disposed on the outer layer. The lubricious coating does not provide structural integrity to the balloon.

The resultant balloons have calculated burst strengths as determined on a 90° bend in the balloon of greater than about 35,000 psi and even more suitably greater than about 40,000 psi.

Dual layer balloons, in contrast, exhibit calculated burst strengths as determined on a 90° bend in the balloon of less than about 35,000 psi.

The resultant balloons suitably have burst pressures of greater than about 400 psi, more suitably greater than about 450 psi, or calculated burst strengths of greater than 45,000 psi, more suitably greater than 47,500 psi and most suitably greater than 50,000 psi. Burst strength is sometimes referred to in the art as hoop strength or radial tensile strength.

Calculated burst strength is different than that of the pressure when the balloon actually bursts during testing of the balloon and takes into account the wall thickness and diameter of the balloon allowing balloons of different wall thicknesses to be compared on the same scale.

Calculated burst strength (psi) is determined using the following formula:

Strength=(P×D/2t)

where P=internal pressure (psi) when the balloon bursts; D is the exterior diameter (mm) of the balloon when a pressure of 10 atmospheres (147 psi) is applied; and t is the wall thickness (mm) of the portion of the balloon with the larger exterior diameter.

Burst strength is improved by adding a thin layer of a lower durometer, softer, more elastic polymer material to the exterior of a dual layer balloon through a mechanism of micro tear resistance without a substantive change balloon wall thickness.

The resultant balloons exhibit substantially the same compliance based on the radial growth of the balloon in millimeters (mm) from nominal pressure to rated burst pressure which is typically a range of about 8 atmosphere (atm) to about 14 atm.

Balloons according to the invention exhibit compliance of no more than about 0.50% radial growth/atmosphere (atm), suitably about 0.25% radial growth/atm to about 0.50% radial growth/atm, more suitably about 0.28% radial growth/atm to about 0.50% radial growth/atm from nominal pressures of about 10 atm to rated burst pressures of about 15 atm to about 30 atm, suitably about 18 atm to about 24 atm which will be illustrated in more detail in the examples below.

The balloon may be formed using any suitable method known in the art. In some embodiments, the method suitably includes forming a tubular parison, stretching the tubular parison, placing the balloon parison in a balloon mold, and forming a balloon by radially expanding the tubular parison into the balloon mold. The balloon is then heat set. Balloon forming with stretching and radial expansion is disclosed in U.S. Pat. Nos. 5,913,861, 5,643,279 and 5,948,345, and in commonly assigned U.S. Pat. Nos. 6,946,092 and 7,1010,597, each of which is incorporated by reference herein in its entirety.

The tubular parison may be formed using coextrusion techniques. The tubular parison has at least three layers including a soft inner layer, a harder intermediate layer, and another soft outer layer.

Alternatively, the softer, more flexible inner and outer layers can be coated either on the balloon parison, or on the balloon itself after it has been formed from the balloon parison.

Coating can be accomplished out of a solvent or solvent blend. The coating can be injected into the tubular parison or balloon, for example.

In some embodiments, it may be desirable for the waist portion of the balloon to be formed of only a single layer. The waist can be masked with an inserted tube, or cleaned after application of the coating.

Suitably, the tubular parison is axially (longitudinally) stretched using a stretching ratio of less than 4.0X where X is the starting length of the tubular parison. In one specific embodiment, the method includes stretching the balloon parison at a ratio of 3.50X wherein X is the starting length of the tubular parison.

At a stretch ratio of significantly more than this, for example, at a stretch ratio of 4.25, a decrease in balloon burst pressure of more than 20% was observed, and the corresponding decrease in calculated burst strength was greater than 10%.

The balloon can then be formed from the tubular parison using any suitable technique including molding. Using molding techniques, the tubular parison can be placed into a mold and radially expanded. Molding pressures may range between about 400 psi and about 600 psi.

Suitably, the balloon is heat set at a temperature of about 150° C. or less. In some embodiments, the heat set temperature is about 125° C. or less. In a specific embodiment, the balloon is heat set at 120° C. It has been found that using a temperature for heat setting that is significantly higher than this, negatively impacts the ultimate burst strength of the balloon. For example, at a heat set temperature of 140° C., the burst pressure was found to decrease by more than 15% over the same balloon formed at 120° C., and the corresponding decrease in burst strength was more than 10%.

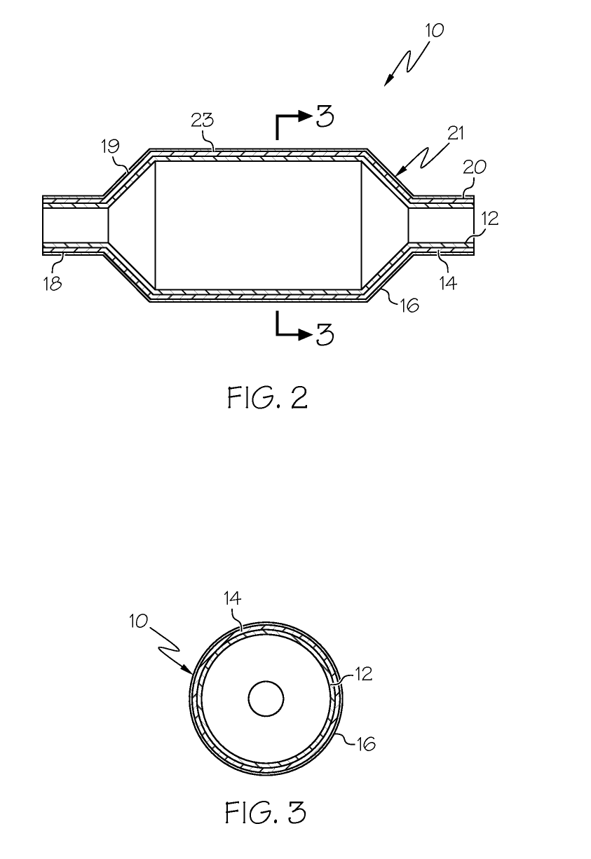

Turning now to the figures, FIG. 2 is a longitudinal cross-sectional representation of a balloon 10 according to the invention. Balloon 10 is shown with three layers having an inner layer 12, an intermediate layer 14 and an outer layer 16 in accordance with the invention. FIG. 3 is a radial cross-section taken at section 3-3 in FIG. 2.

The balloon can further include a lubricious coating (not shown). The lubricious coating may be applied to the balloon waists 18, 20, balloon cones 19, 21 and balloon body 23, or any portion thereof. Suitably, lubricious coatings are applied at a thickness of about 0.1 microns to about 5.0 microns, more suitably about 0.5 microns to about 2.0 microns.

Any suitable lubricious material may be employed in the lubricious coating. Such lubricious coatings are known in the art. Examples of materials that can be used in the lubricious coatings include both thermoplastic and thermoset materials. The lubricious polymers can be either hydrophobic or hydrophilic. Hydrophilic materials are often preferred because they are typically more biocompatible. Lubricious coatings are disclosed in commonly assigned U.S. Pat. No. 5,509,899, the entire content of which is incorporated by reference herein.

Interpenetrating polymer networks can also be employed. These materials are described, for example, in commonly assigned U.S. Pat. No. 5,693,034, the entire content of which is incorporated by reference herein.

Coatings for the controlled delivery of therapeutic agents may also be optionally added.

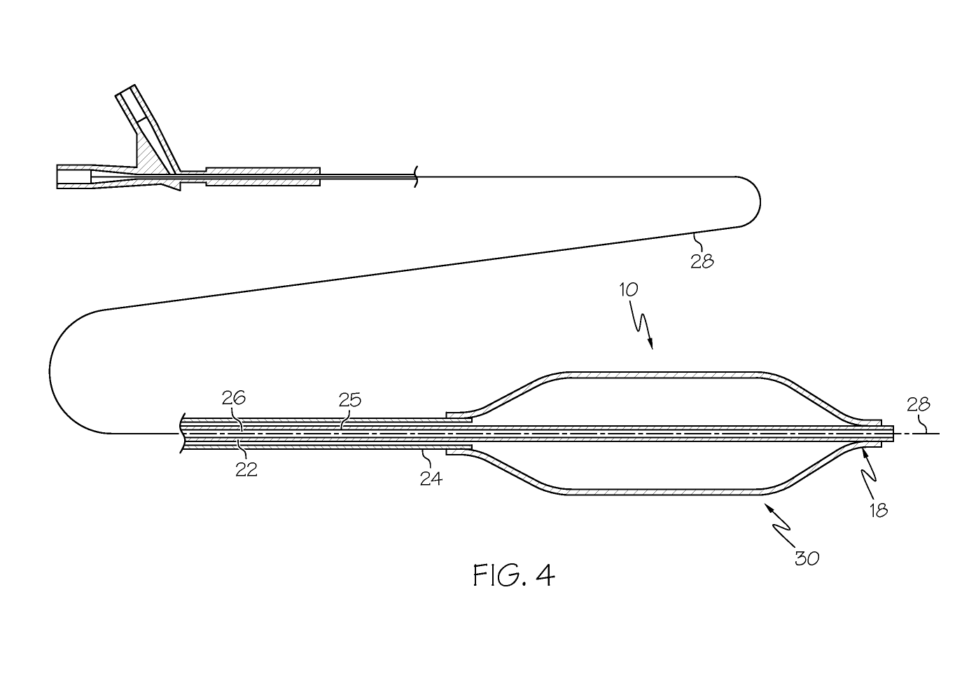

FIG. 4 is a longitudinal cross-section of a catheter assembly 30 equipped with a balloon 10 according to the invention. Catheter assembly 30 is a dual-lumen catheter having an inner shaft 22 and an outer shaft 24. Inner shaft 22 has an inner surface 25 defining a guide wire lumen 26. Guide wire 28 is shown disposed within lumen 26.

Proximal waist 18 of balloon 10 is disposed about the distal end of outer shaft 24 and distal waist 20 of balloon 10 is disposed about the distal end of inner shaft 22.

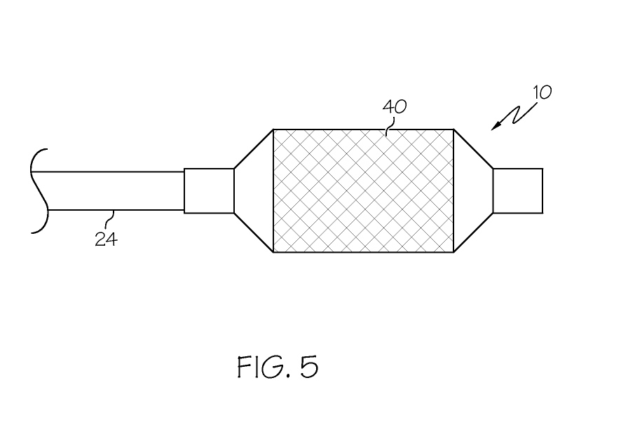

The assembly may further incorporate a stent 40 disposed about balloon 10 as shown in FIG. 5. In the case of stent delivery application, it may be desirable to have a lubricious coating applied to only the waist portions 18, 20, cone portions 19, 21, or a combination of the waist and cone portions.

The balloons described herein may be employed in any of a variety of medical procedures including, but not limited to, angioplasty (PTCA) procedures, for delivery of medical devices such as stents (SDS), genito-urinary procedures, biliary procedures, neurological procedures, peripheral vascular procedures, renal procedures, and so forth.

The following non-limiting examples further illustrate some aspects of the present invention.

### EXAMPLES

### Example 1

Pebax® 7233 (inner layer), Vestamid L2101F (intermediate layer) and Pebax® 7033 (outer layer) were coextruded axially into the tubing of ID 0.04810 by OD 0.08970 inches. The inner Pebax® 7233 layer had a wall thickness of 0.0077″, the intermediate Vestamid® L2101F layer had a wall thickness of 0.0112″ and the outer Pebax® 7033 layer had a wall thickness of 0.0019″. The cross-sectional ratio of each of the layers was approximately 40% inner layer/50% intermediate layer/10% outer layer.

The tube was stretched at the speed of 50 mm/sec at 45° C. temperature with the inside pressure of 400 psi at a stretch ratio of 3.50. The stretched tube was inserted into a 0.1260 inch balloon mold (inner diameter or ID or balloon mold), and a balloon was formed at 95° C. and heat set right after formation at 120° C. for 1 minute. The balloon forming pressure was 500 psi.

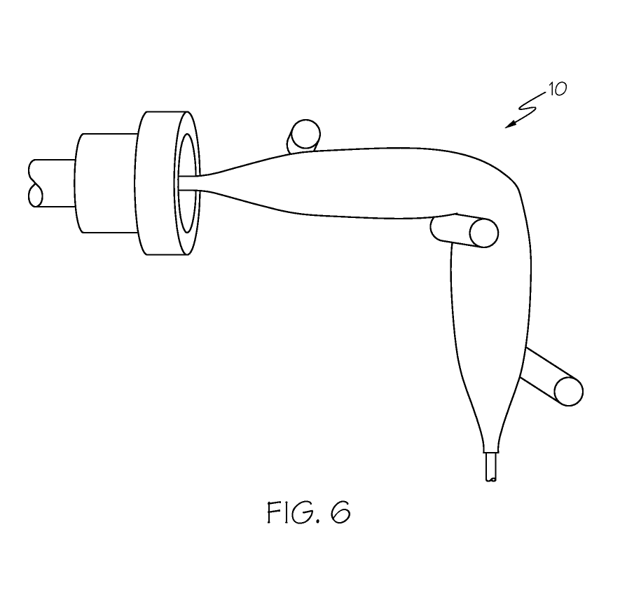

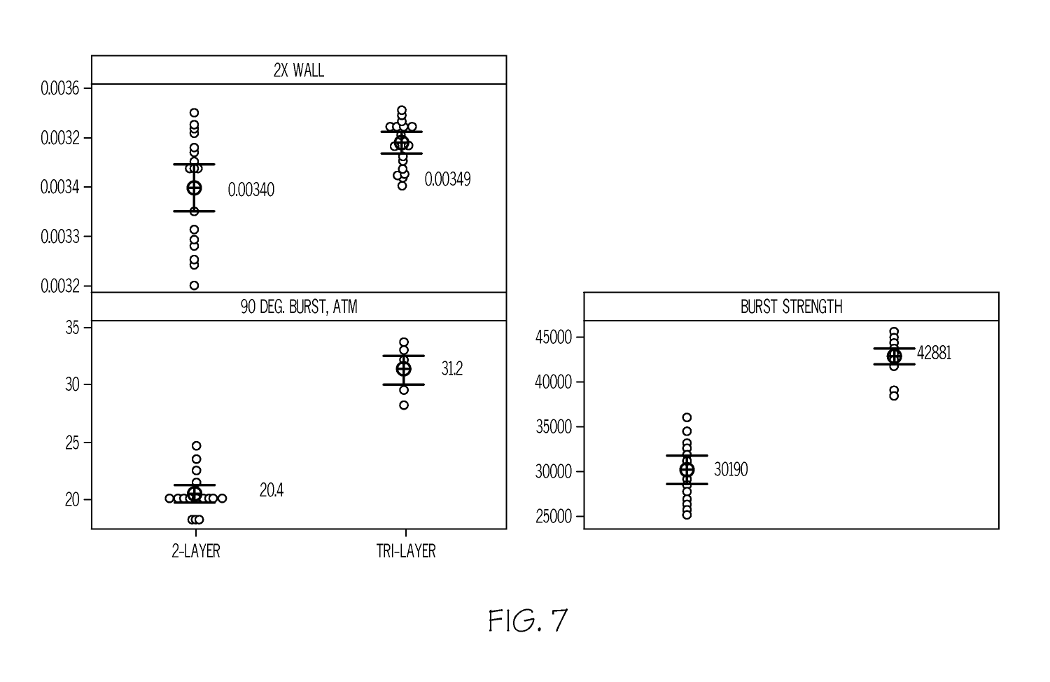

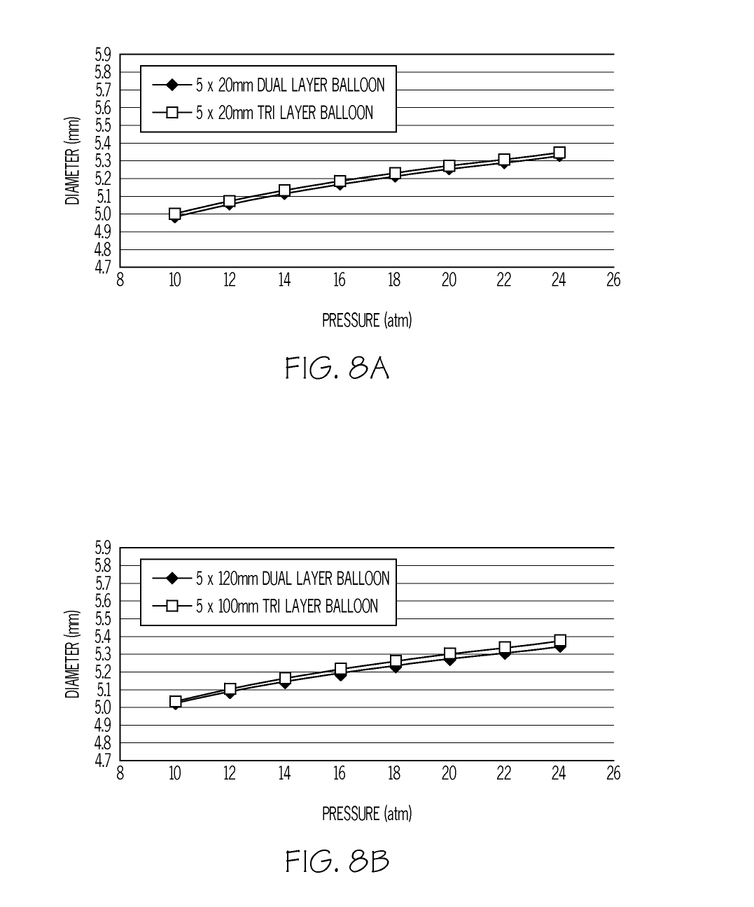

The average balloon burst for the tri-layer balloon as determined on a 90 degree bend in the balloon (See FIG. 6) at 458.5 psi (31.2 atm) at a double wall thickness of 0.00340 inches while a comparably formed dual layer having with the same inner and intermediate layer but no outer layer exhibited an average balloon burst as determined on a 90° bend in the balloon was about 300 psi (20.4 atmosphere).

The calculated burst strength (psi) was then determined using the following formula:

Strength=(P×D/2t)

where P=internal pressure (psi) when the balloon bursts; D is the exterior diameter (mm) of the balloon when a burst pressure of 10 atmospheres (147 psi) is applied; and t is the wall thickness of the portion of the balloon with the larger exterior diameter.

The calculated burst strength of the tri-layer balloon was 42,881 psi while the calculated burst strength of the dual layer balloon was 30,190 psi.

This example is illustrated by FIG. 7.

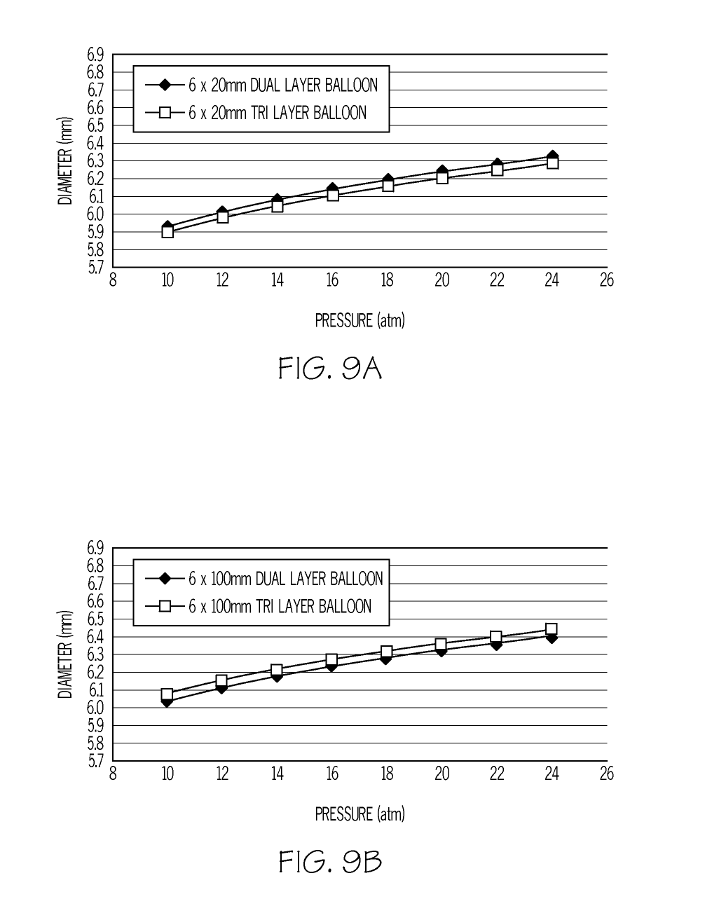

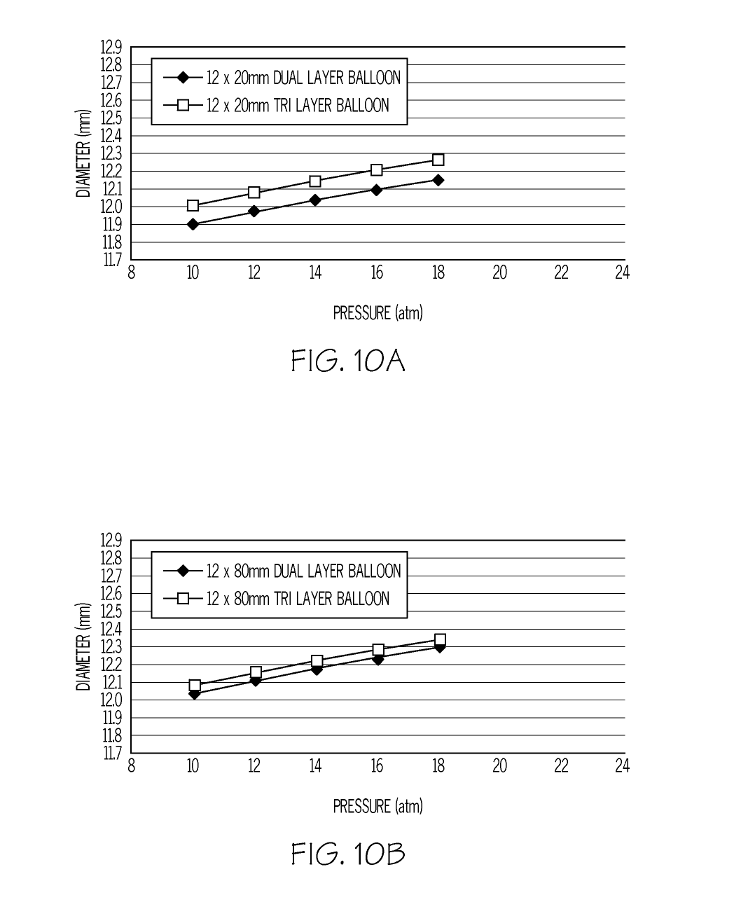

The compliance of the tri-layer balloon is substantially the same as that of the dual-layer balloon as determined by the radial growth in mm from nominal pressure to rated burst pressure for balloons of various sizes as shown in FIGS. 8A-10B. The compliance for the balloons is about no more than about 0.50% radial growth/atm from nominal pressure to rated burst pressure, suitably about 0.25% radial growth/atm to about 0.50% radial growth/atm from nominal pressure to rated burst pressure, more suitably about 0.28% radial growth/atm to about 0.50% radial growth/atm from nominal pressure to rated burst pressure.

All published documents, including all US patent documents and US patent publications, mentioned anywhere in this application are hereby expressly incorporated herein by reference in their entirety. Any copending patent applications, mentioned anywhere in this application are also hereby expressly incorporated herein by reference in their entirety. Citation or discussion of a reference herein shall not be construed as an admission that such is prior art.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

1. An expandable medical balloon comprising:
an inner layer formed of a poly(ether-block-amide) copolymer;
an intermediate layer formed of a polyamide; and
an outer layer comprising a polymeric material having a flexural modulus that is less than the intermediate layer;
wherein the calculated burst strength of the balloon determined on a 90 degree bend in the balloon is about 35,000 psi or higher.

2. The expandable medical balloon of claim 1 wherein the burst strength of the balloon determined on a 90 degree bend in the balloon is about 40,000 psi or higher.

3. The expandable medical balloon of claim 1 wherein the compliance of the balloon is substantially the same as that of a balloon comprising only an inner layer formed of poly(ether-block-amide) and an outer layer formed of polyamide.

4. The expandable medical balloon of claim 1 wherein the compliance of the balloon is no more than about 0.50% radial growth/atmosphere.

5. The balloon of claim 1 wherein the outer layer comprises poly(ether-block-amide).

6. The balloon of claim 1 wherein the inner layer and outer layer comprise a flexural modulus of about 50,000 psi to about 110,000 psi.

7. The balloon of claim 1 wherein the inner and outer layer comprise a flexural modulus of about 55,000 psi to about 75,000 psi.

8. The balloon of claim 1 wherein the intermediate layer comprises a flexural modulus of about 130,000 psi to about 230,000 psi.

9. An expandable medical balloon comprising:
an inner layer formed of a poly(ether-block-amide) copolymer;
an intermediate layer formed of a polyamide; and
an outer layer comprising a polymeric material having a flexural modulus that is less than the flexural modulus of the intermediate layer outer layer;
wherein the compliance is about 0.25% radial growth/atmosphere to about 0.50% radial growth/atmosphere from nominal pressure to rated burst pressure

10. The expandable medical balloon of claim 9 wherein the calculated burst strength determined on a 90 degree bend in the balloon is about 35,000 psi or higher.

11. The expandable medical balloon of claim 9 wherein the calculated burst strength determined on a 90 degree bend in the balloon is about 40,000 psi or higher.

12. The expandable medical balloon of claim 9 wherein the compliance is substantially the same as that of a balloon comprising only an inner layer formed of poly(ether-block-amide) and an outer layer formed of polyamide.

13. The expandable medical balloon of claim 9 wherein the durometer of the inner and outer layers is about 25D to about 75D on the Shore D hardness scale.

14. The expandable medical balloon of claim 9 wherein the durometer of the intermediate layer is about 60 to about 115 on the Rockwell hardness scale.

15. The expandable medical balloon of claim 9 wherein the maximum compliance of the balloon is no more than about 0.5%/atmosphere from nominal pressure to rated burst pressure.

16. The expandable medical balloon of claim 9 wherein the outer layer is formed from a poly(ether-block-amide).

17. An expandable medical balloon comprising:
an inner layer formed of a polymer material having a first flexural modulus;
an intermediate layer formed of a material having a second flexural; and
an outer layer formed of a material having a third flexural modulus;
wherein the flexural modulus of the inner layer and outer layer is 50,000 psi to about 110,000 psi, the flexural modulus of the intermediate layer is about 130,000 to about 230,000 psi and the calculated burst strength of the balloon determined on a 90 degree bend in the balloon is about 35,000 psi or higher.

18. The expandable medical balloon of claim 17 wherein the compliance of the balloon is about 0.25% radial growth/atmosphere to about 0.50% radial growth/atmosphere from nominal pressure to rated burst pressure.

19. The expandable medical balloon of claim 17 wherein the burst strength of a balloon determined on a 90 degree bend of the balloon is greater than about 40,000 psi.

20. The expandable medical balloon of claim 17 wherein the inner layer and outer layer are formed from a poly(ether-block-amide) and the inner layer is formed from nylon 12.
